SEQUENCE LISTING (8 sequences)

SEQ ID NO: 1 - protein, 591 aa, artificial sequence
  Reference NS5B
SMSYTWTGALITPCAAEESKLPINALSNSLLRHHNMVYATTSRSASQRQKKVTFDRLQVLDNHYQDVLKEMKAKASTVKARLLSVEEACKLTPPHSAKSKFGYGAKDVRNLSSKAVNHIRSVWEDLLEDTDTPIDTTIMAKNEVFCVQPEKGGRKPARLIVFPDLGVRVCEKMALYDVVSTLPQAVMGSAYGFQYSPGQRVEFLVNAWKSKKTPMGFAYDTRCFDSTVTESDIRVEESIYQCCDLAPEARQAIRSLTERLYVGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRARLLSQGGRAATCGKYLFNWAVKTKLKLTPIPAASQLDLSGWFVAGYSGGDIYHSLSRARPRWFMLCLLLLSVGVGIYLLPNR

SEQ ID NO: 2 - protein, 130 aa, artificial sequence
  216-345 NS5B not mutated at Day 0
GVPYDTRCFDSTVTENDIRVEESIYQCCDLAPEARQAIRSLTERLYVGGPLTNSKGQNCGYRRCRASGVLTTSCGNTLTCYLKATAACRAAKLQDCTLLVNGDDLVVICESAGTQEDAANLRVFTEAMTR

SEQ ID NO: 3 - protein, 130 aa, artificial sequence
  216-345 NS5B mutated at Day 21
GVPYDTRCFDSTVTENDIRVEESIYQCCDLAPEARQAIRSLTERLYAGGSLTNSKGQNCGYRRCRASGVLTTSCGNTLTCYLKATAACRAAKLQDCTLLVDGDDLVVICESAGTQEDAANLRVFTEAMTR

SEQ ID NO: 4 - DNA, 390 bp, artificial sequence
  216-345 NS5B not mutated at Day 0
ggggtcccgtatgatacccgctgctttgactcaacggtcactgagaatgacatccgtgttgaggagtcaatttaccaatgttgtgacttagcccccgaagccagacaggctataaggtcgctcacggagcggctttatgtcgggggccccctgactaattcaaaagggcagaactgcggttatcgccggtgccgcgcaagcggcgtgctgacgaccagctgcggtaataccctcacatgttacttgaaggccactgcggcctgtcgagctgcaaagctccaggactgcacattgctcgtgaacggcgacgaccttgtcgttatctgcgaaagcgcgggaacccaagaggacgcggcgaacctacgagtcttcacggaggctatgaccagg

SEQ ID NO: 5 - DNA, 390 bp, artificial sequence
  216-345 NS5B mutated at Day 21
ggggtcccgtatgatacccgctgctttgactcaacggtcactgagaacgacatccgtgttgaggagtcaatttaccaatgttgtgacttagcccccgaagccagacaggctataaggtcgctcacagagcggctttatgccgggggctccctgactaattcaaaagggcagaactgcggttatcgccggtgccgcgcgagcggcgtgctgacgactagctgcggtaataccctcacatgttacttgaaggccactgcggcctgtcgagctgcgaagctccaggactgcacattgctcgtggacggcgacgaccttgttgttatctgcgaaagcgcgggaactcaagaggacgcggcgaacctacgagtcttcacggaggctatgaccagg

SEQ ID NO: 6 - DNA, 21 bp, artificial sequence
  2CH primer
aactactgtcttcacgcagaa

SEQ ID NO: 7 - DNA, 21 bp, artificial sequence
  1TS primer
gcgacccaacactactcggct

SEQ ID NO: 8 - DNA, 19 bp, artificial sequence
  TM416 primer
aacccgctcaatgcctgga

(19) United States
(12) Patent Application Publication
(10) Pub. No.: US 2011/0091969 A1
(43) Pub. Date: Apr. 21, 2011

(54) MUTATIONS IN THE NS5B PROTEIN OF THE HCV

(75) Inventors: Emmanuel Drouet, Corenc (FR); Yassine Rechoum, Houston, TX (US); Florence Fouque, Le Rove (FR)

(73) Assignees: UNIVERSITE JOSEPH FOURIER, Grenoble (FR); INSTITUT PASTEUR, Paris (FR)

(21) Appl. No.: 12/996,521

(22) PCT Filed: Jun. 25, 2009
(86) PCT No.: PCT/FR2009/051222

§ 371 Date: Dec. 6, 2010

(30) Foreign Application Priority Data
Jun. 26, 2008 (FR) 0854292

(51) Int. Cl. C12N 5/10; C07K 14/00; C07H 21/00; C12N 15/00

(52) U.S. Cl. 435/348; 530/350; 536/23.72; 536/23.5; 435/320.1

(57) ABSTRACT

An NS5B protein of the hepatitis C virus (HCV), with good replication performance, has a point mutation in at least one of the following positions:
in the position corresponding to residue 262 of SEQ ID NO: 1; and/or
in the position corresponding to residue 265 of SEQ ID NO: 1; and/or
in the position corresponding to residue 316 of SEQ ID NO: 1.

### TECHNICAL FIELD

This invention concerns the identification of new mutations present in the NS5B protein of the hepatitis C virus (HCV) amplified in mosquitoes of the Aedes genus.

The identification of such mutations, associated with effective replication, offers promising prospects particularly for replicons.

### PRIOR ART

The hepatitis C virus (HCV) has been identified as being responsible for non-A non-B developed hepatitis, frequently evolving into chronic malignant pathologies such as cirrhosis of the liver or even hepatocellular carcinoma. It has been estimated that approximately 3% of the world's population is infected by HCV, with an increase in infections of 3 to 4 million people per year (Neumayr et al., 2000).

HCV is a member of the Hepacivirus genus of the Flaviviridae family which are enveloped single-stranded RNA viruses, and include the viruses responsible for major epidemic diseases such as yellow fever (YF), dengue fever (DEN) and dengue haemorrhagic fever (DHF), Japanese encephalitis (JE), Saint-Louis encephalitis (SLE), West Nile fever (WN), to mention only the most important.

The majority of Flaviviruses are transmitted by insect vectors, by very different epidemiological methods. Certain diseases are typically human and never affect animals such as DEN and DHF. Other infections are zoonotic instead, and more or less accidentally affect humans, such as JE, SLE and WN. Finally, certain Flaviviruses can circulate epidemically both in human and animal populations (WN). These different epidemiological methods nevertheless have common fundamental factors such as viral amplification in insect cells.

As far as HCV is concerned, the transmission routes, which are well established today, are essentially: transmission via blood products, through drug addiction, and in a nosocomial context. Nevertheless, 30 to 40% of HCV infections recorded nowadays in humans have no explained origin, in as far as none of the conditions listed has been met (Neumayr et al., 2000).

The HCV genome is composed of a single strand of RNA of approximately 9.6 kilobases, which codes for a polyprotein precursor of approximately 3,000 amino acids. This polyprotein is composed of viral proteins in the following order: C-E1-E2-p7-NS2-NS3-NS4A-NS4B-NS5A-NS5B.

Among the non-structural proteins, NS5B is an RNA-dependent RNA polymerase (RdRp), described for example in the document WO 96/37619.

The effective replication of HCV in cell culture has been associated with adaptive mutations, identified particularly in the gene encoding NS5B, which greatly increase the effectiveness of replication. Thus, the document WO 2005/047463 describes 3 mutations (serine in position 24, isoleucine in position 31 and leucine in position 392) in NS5B in a genotype 2b HCV, increasing the effectiveness of a replicon integrating this sequence.

However, in the fight against type C viral hepatitis, a major obstacle impeding the development of a treatment and effective vaccine is the lack of a system allowing high-level multiplication of the virus in vitro. There is therefore a persistent need to develop the systems, particularly more effective replicons.

### DESCRIPTION OF THE INVENTION

The applicant has succeeded in obtaining in vivo amplification of the hepatitis C virus (HCV) in mosquitoes of the Aedes genus.

The viral load used comes preferably from a serum, to advantage of human origin, e.g. that of a subject with chronic hepatitis caused by HCV of genotype 1b.

After amplification lasting for a mean of 21 days, the analysis of the HCV genome, which had been effectively replicated, revealed the presence of mutations in NS5B.

Thus, according to a first aspect, this invention concerns an NS5B protein of a mutated hepatitis C virus (HCV) with good replication performance.

“HCV NS5B protein” is taken to mean the RNA-dependent RNA polymerase (RdRp) encoded by the 3′ end of the region encoding the viral genome.

Although liable to variation, this protein has been characterised as consisting of 591 residues or amino acids. This size is particularly conserved in NS5B proteins from viruses of genotype 1b or 2b described in the prior state of the art mentioned above.

The sequence available in the databases under the number EF507504 was selected as a reference. It corresponds to an entire genotype 1b genome. The C-terminal region of the corresponding polyprotein contains the NS5B protein with the identified sequence SEQ ID NO: 1 of this application.

Thus and for the invention, by “HCV NS5B protein” we mean to advantage a protein at least 70% identical, to advantage 80%, 85%, 90%, or even 95% identical with the sequence SEQ ID NO: 1. To advantage, it is functionally equivalent to sequence SEQ ID NO: 1, for which the enzyme activity can be easily tested using the test described in the Lohmann et al document dated 1997.

Thus, it has been determined that a protein of interest according to the invention would have a mutation in at least one of the following positions:
in the position corresponding to residue 262 of SEQ ID NO: 1; and/or
in the position corresponding to residue 265 of SEQ ID NO: 1; and/or
in the position corresponding to residue 316 of SEQ ID NO: 1.

It should be noted that, these mutations have the particular feature of being located in the active site of this protein. Indeed, Lohmann et al. (1997) showed 4 amino acid patterns which were essential for the activity of HCV NS5B polymerase. In particular, it was shown that replacing aspartic acid (Asp or D) in position 220, glycine (Gly or G) in position 283 or aspartic acid (Asp or D) in position 318 completely destroyed the enzyme activity. It can be seen that the 3 residues targeted by this invention are situated within this crucial region.

In practice, this definition covers:
3 mutants (262; 265; 316);
3 double mutants (262+265; 262+316; 265+316);
1 triple mutant (262+265+316).

To advantage, the single mutant (I262) with an isoleucine (I262) in the position corresponding to residue 262 of SEQ ID NO: 1 is excluded.

Similarly, the double mutant (I262/C316) with an isoleucine (I316) in the position corresponding to residue 262 of SEQ ID NO: 1 and a cysteine in the position corresponding to residue 316 of SEQ ID NO: 1 is excluded.

To advantage, the residue in the position corresponding to residue 262 of SEQ ID NO: 1 is not an amino acid selected from the following group: valine (Val or V), leucine (Leu or L), isoleucine (Ile or I) and cysteine (Cys or C), in particular isoleucine (Ile or I). To even greater advantage, the residue in the position corresponding to residue 262 of SEQ ID NO: 1 is an alanine residue (Ala or A).

To advantage, the residue in the position corresponding to residue 265 of SEQ ID NO: 1 is not proline (Pro or P). To still greater advantage, it is a serine (Ser or S).

To advantage, the residue in the position corresponding to residue 316 of SEQ ID NO: 1 is not an amino acid selected from the following group: asparagine (Asn or N), cysteine (Cys or C) and histidine (His or H), in particular cysteine (Cys or C). To even greater advantage, the residue in the position corresponding to residue 316 of SEQ ID NO: 1 is an aspartic acid or aspartate residue (Asp or D).

It appears that in the sequence SEQ ID. NO: 1, the residue in position 262 is valine (Val or V), the residue in position 265 is proline (Pro or P) and the residue in position 316 is asparagine (Asn or N).

These residues are also found in positions 47, 50 and 101 of the sequence SEQ ID NO: 2 corresponding to the residues 216 to 345 of NS5B encoded by the viral genome which served for the infection on day 0 (D0), even if elsewhere point differences are seen.

The expression “position corresponding to the residue” indicates that the exact numbering of the amino acids can vary depending on the sequences. Thus, a corresponding position can be identified by aligning the sequences to obtain the highest degree of homology around the position concerned.

Indeed, it should be noted that the NS5B sequences reported in the literature certainly show strong homology but also point differences.

Thus, in the reference document WO 96/37619, and more precisely in the sequence SEQ ID NO: 1 of this document which corresponds to NS5B, the residues in positions 265 and 316 are indeed proline and asparagine respectively. On the other hand, position 262 corresponds to isoleucine and not valine, but is not alanine as in this invention.

As far as the reference document WO 2005/047463 is concerned, and more precisely in the sequence SEQ ID NO: 1 of this document which corresponds to NS5B from a genotype 2b HCV, the residues in positions 262 and 265 are indeed valine and proline respectively. On the other hand, position 316 corresponds to cysteine and not asparagine, but is not aspartic acid as in this invention

To the applicant's knowledge, this is the first time that mutations in the particular positions identified have been reported.

According to an advantageous embodiment, the NS5B protein according to the invention has the three mutations described above.

In fact, when the regions from amino acid 216 to 345 of NS5B are compared between day 0 (D0) and day 21 (D21), i.e. by comparing the sequences SEQ ID NO: 2 and SEQ ID NO: 3, only the three residues in these three positions differ.

Thus, according to a preferred embodiment, an NS5B protein according to the invention contains the sequence SEQ ID NO: 3. In other words, starting from a “wild” NS5B protein, it is possible to replace the residues in positions 262, 265 and 316 by alanine, serine and aspartic acid respectively, or replace the region 216-345 by the sequence SEQ ID NO: 3.

Fragments of the NS5B protein are also targeted by the invention, the said fragments containing at least one point mutation at one of the three key positions identified for this invention. To advantage, they are functional fragments the RdRp activity of which can be easily tested using the test described in the Lohmann et at document dated 1997.

Another aspect of the invention concerns a sequence of nucleic acids encoding an NS5B protein mutated as described above.

“Nucleic acid” is taken to mean either a DNA or an RNA molecule. The nucleotide sequences shown in this invention correspond to DNA but the corresponding RNA sequences are easily deduced by those working in the field by replacing T by U.

Moreover, a nucleic acid sequence according to the invention could just as well correspond strictly to the part encoding NS5B as to an entire HCV genome containing the mutations defined above.

To advantage, the codon encoding the residue corresponding to NS5B position 262 must encode alanine and can therefore be GCA, GCC, GCG or GCT.

To advantage, the codon encoding the residue corresponding to NS5B position 265 must encode serine and can therefore be TCA, TCC, TCG, TCT, AGC or AGT.

To advantage, the codon encoding the residue corresponding to NS5B position 316 must encode aspartic acid and can therefore be GAC or GAT.

As an illustration, the sequence encoding the portion 216 to 345 of HCV NS5B on day 0 (D0) appears as the sequence SEQ ID NO: 4.

In comparison, the sequence encoding this same portion on day 21 (D21) appears as the sequence SEQ ID NO: 5.

This can be seen in positions 139 to 141 of SEQ ID NO: 4 and 5 (encoding the residue in position 47 of sequences SEQ ID NO: 2 and 3 or in position 262 of the sequence SEQ ID NO: 1), the GTC codon (SEQ ID NO: 4) encoding valine becomes a GCC codon (SEQ ID NO: 5) encoding alanine

Similarly, in positions 148 to 150 of SEQ ID NO: 4 and 5 (encoding the residue in position 50 of sequences SEQ ID NO: 2 and 3 or in position 265 of the sequence SEQ ID NO: 1), the CCC codon (SEQ ID NO: 4) encoding proline becomes a TCC codon (SEQ ID NO: 5) encoding serine.

Finally, in positions 301 and 303 of SEQ ID NO: 4 and 5 (corresponding to position 101 of sequences SEQ ID NO: 2 and 3 or in position 316 of the sequence SEQ ID NO: 1), the AAC codon (SEQ ID NO: 4) encoding asparagine becomes a GAC codon (SEQ ID NO: 5) encoding aspartic acid.

Typically, a nucleic acid sequence according to the invention can thus include the sequence SEQ ID NO: 5.

To advantage, the proteins, nucleic acid sequences and fragments of them described in this invention are of human origin.

For this application, 3 adaptive mutations are described which are likely to improve HCV replication. The sequences brought to light have many applications, particularly for vectors or replicons.

Thus, another aspect of the invention concerns a vector including a nucleic acid sequence according to the invention. To advantage, this sequence is put under the control of regulator sequences able to ensure its expression, particularly a promoter. In particular, this embodiment allows a mutated NS5B protein to be produced containing at least one, two or even three of the mutations identified above.

According to another aspect, it is a replicon containing a sequence according to the invention. An HCV replicon is a nucleic acid molecule capable of autonomously replicating in cell culture and producing detectable levels of one or more viral proteins. The HCV replicon expresses the components of the replication machinery derived from HCV and contains elements required for replication in cell culture.

HCV replicons already exist, such as the one described in document EP 1 666 598. It is therefore possible to modify the sequence encoding NS5B in this replicon in the positions and using the mutations described above. Alternately, the region of the replicon encoding NS5B can be replaced by a sequence according to the invention. A chimeric replicon is then obtained. It is thus possible to increase the efficacy of already existing replicons.

Host cells containing vectors or replicons as previously defined are also targeted by the invention. Preferably, these cells come from mosquitoes, to advantage from the Aedes genus. Without wishing to be bound to any given theory, it is supposed that these mutations resulting from selection in the mosquito, the combination of this host and the mutated NS5B protein according to the invention should give rise to a particularly effective replication system.

These in vitro replication systems provide many prospective uses. They allow the ability of a compound to inhibit replication activity to be tested and therefore make screening tools for anti-HCV therapeutic molecules.

### EXAMPLES OF EMBODIMENTS

The invention and the advantages resulting from it will be better illustrated by the following examples of embodiments and the attached figures. However, these examples are in no case limiting.

FIG. 1 shows the competence of mosquitoes for HCV replication, 21 days after contact of the mosquito with the virus. The competence depends on the genus of the mosquito: the HCV genome was detected by qRT-PCR 21 days after incubation with the virus in the mosquitoes of the Aedes genus but not in those of the Culex genus.

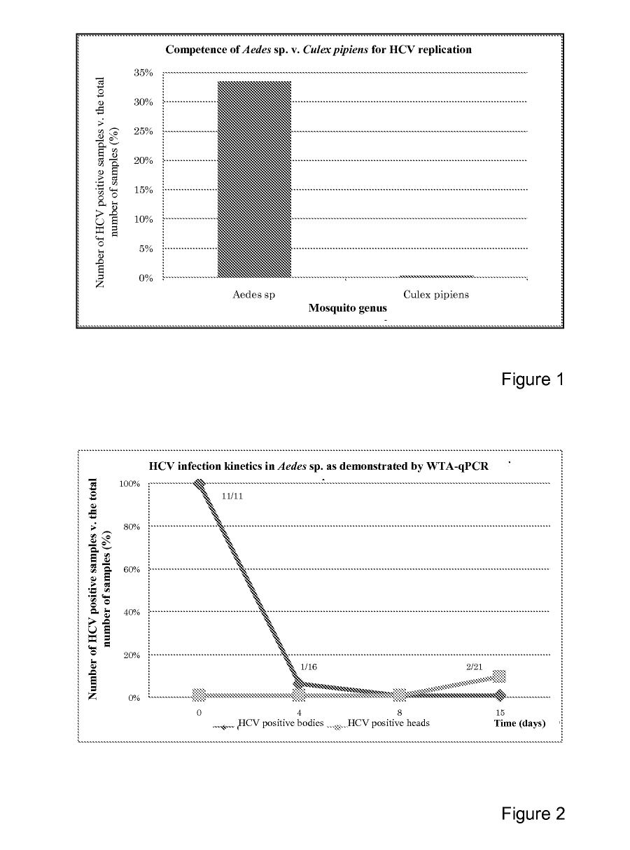

FIG. 2 shows the development of detection of the viral genome in the bodies and heads of the mosquitoes. The total RNA extracts were analysed after qRT-PCR, preceded by whole transcriptome amplification (WTA). From the 15^{th} day, the WTA-qPCR combination revealed the presence of the HCV genome in the head of the mosquitoes.

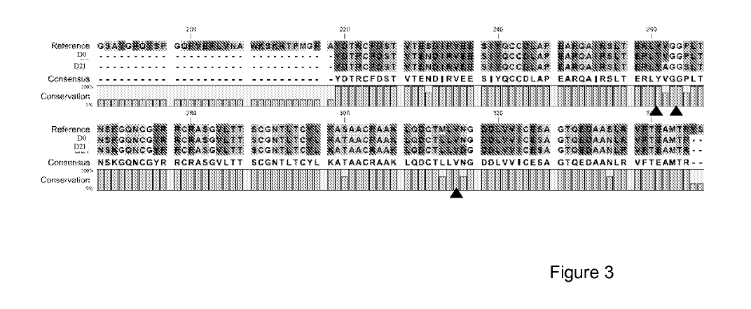

FIG. 3 represents alignment of sequences of the NS5B amplified region. Region NS5B 8228-8628 of positive mosquitoes was sequenced on day 21 (D21) and compared with that of infectious mosquitoes on day 0 (D0). The NS5B sequence corresponding to sequence EF407504 served as a reference. The triangles show the amino acids mutated on day 21 (262 Val/Ala, 265 Pro/Ser, 316 Asn/Asp). Sequence analysis concerned 2 regions of the viral genome because they are frequently sequenced and are phylogenetically very informative: the 5′UTR region is a highly conserved region whereas the NS5B gene has been reported as being particularly affected by adaptive mutations during the acute phase of infection (Kuntzen et al., 2007).

### EXPERIMENTAL PART

### I—Experimental Infections

### 1. Breeding the Species Tested

The individuals tested come from breeding from mosquitoes (eggs, larvae and adults) collected in the field in the urban area of Marseilles for the species Culex pipiens and in the Tour du Valet en Camargue reserve for the species Aedes vexans and Aedes caspius. The breeding took place in the ENSAM premises situated in the Domaine du Merle, in Salon de Provence. Breeding was in a classic insectarium complying with standards for breeding. The larvae were fed with brewer's yeast and fish food and the adults were fed with a sucrose solution. The Culex and Aedes blood meals were composed of washed sheep red cells, the objective of washing being to eliminate interference from blood components.

### 2. Introduction of the Mosquitoes into the Secure Laboratory (Level 2+)

On day D-1, the female mosquitoes were divided into groups, placed in the engorgement boxes with moist cotton-wool on each box and introduced into the secure laboratory. The mosquitoes were kept in controlled temperature and humidity conditions (T=25° C.±2, RH 80%±10).

### 3. Introduction of the HCV into the L2+Laboratory

On day D0, the viral solution containing the HCV virus (serum from a patient with chronic hepatitis at 2^{E}6 copies/ml) was brought by special carrier to the L2+ where the mosquitoes had been put. The viral solution was provided by the virology laboratory of the Albert-Michallon university hospital in Grenoble. The viral solution was kept in the L2+ freezer at −80° C.

### 4. Preparation of the Infected Blood Solution

On day D0, the washed non-infected red cells (sheep's blood) and the viral solution were mixed to obtain a sufficient titre to infect the mosquitoes (˜1500 copies/mosquito). ATP was added to make the blood more attractive and increase the desire of the females for it. Each mosquito takes between 3 and 5 μl per blood meal.

### 5. Infection of the Groups of Females

On day D0, the engorger containing the infected blood, warmed to 37° C., was presented to the females which had been deprived of glucose for 24 hours. The engorger was placed on each box and left there for about 4 hours for blood meals to be taken. In parallel, an engorger containing non-contaminated blood was presented to batches of negative control females. At the end of the period of engorgement of the females, they were collected using a mouth aspirator, anaesthetised using chloroform vapour and sorted under a binocular microscope. Females that had engorged themselves with blood were put back into the cages. An artificial breeding area was left in each metal cage for the females to lay (water trough for the Aedes females and moist earth for the Culex females). From this day the metal cages containing the engorged females were not opened or handled until the mosquitoes were sacrificed.

### 6. Care for the Groups of Females

The groups of females were kept at a constant temperature of about 28° C. with controlled humidity of at least 70%. Each day, the females were fed via cotton soaked in a solution of 10% glucose put onto the metal cage. The laying areas were moistened through the cage with a wash bottle.

### 7. Treatment of the Females

On the sampling days, the mosquitoes were sacrificed by putting the metal cage in the cold. The mosquitoes were then put individually into 1.5 ml tubes, labelled and put into the freezer until the RNA extraction stage before the PCRs. The negative controls, engorged with blood not containing viruses were also sacrificed. The group of mosquitoes, sacrificed on day D0 and put into the freezer at −80° C., constituted the D0 positive controls of engorgement, extraction and amplification of the viral RNA.

### II—Detection of Viral RNA

### 8. Extraction of the RNA for the Infected Organs

The extraction of the HCV RNA from mosquito tissues was performed in a hood, using the “High Pure RNA Tissue Kit” marketed by Roche.

### 9. Amplification of the Viral RNA

The primers used as well as the probe (for the qPCR) were designed to amplify the conserved region at the 5′ non-coding end of the viral genome. We used the sense primer 2CH (5′-AAC TAC TGT CTT CAC GCA GAA-3′) (SEQ ID NO: 6), located between nucleotides-289 and -269, and the antisense primer 1TS (5′ GCG ACC CAA CAC TAC TCG GCT-3′) (SEQ ID. NO: 7), located between nucleotides-70 and -90. The probe used for the qPCR was TM416 (5′-6Fam-AAC CCG CTC AAT GCC TGG A-Tamra-3′) (SEQ ID. 8) situated between nucleotides-137 and -119.

The composition of the reaction mixture for the classic RT-PCR and the qRT-PCR is summarised in the following table:

| | Classic RT-PCR | | | RT-PCR in real time | | |
|---|---|---|---|---|---|---|
| | | | Volume per | | | Volume per |
| | Initial conc. | Final conc. | tube (μl) | Initial conc. | Final conc. | tube (μl) |
| Buffer | 5 X | 1 X | 12.5 | 5 X | 1 X | 4.8 |
| dNTP | 10 mM | | 2 | 10 mM | | 0.8 |
| Sense primer | 10 μM | 0.6 μM | 0.75 | 10 μM | 0.6 μM | 0.3 |
| Antisense | 10 μM | 0.6 μM | 0.75 | 10 μM | 0.6 μM | 0.3 |
| primer | | | | | | |
| Probe | — | — | — | 25 μM | 0.312 μM | 0.05 |
| Water qs final | — | — | 19.8 | — | — | 7.95 |
| volume | | | | | | |
| RNase OUT | | | 1.2 | | | 0.4 |
| RNA master | | | 3 | | | 1.2 |
| Mix | | | | | | |
| Matrix (RNA) | — | — | 10 | — | — | 4 |

| | |
|---|---|
| Final volume: 50 μl | Final volume: 20 μl |

The RT-PCR was performed under the following conditions: retrotranscription occurred for 30 minutes at 50° C., followed by a stage deactivating the reverse transcriptase (15 minutes at 95° C.), then 45 PCR cycles alternating an elongation stage (1 minute at 63° C.) and a denaturation stage (30 seconds at 90° C.).

### 10. Sequencing of the Region (52-271) Corresponding to the IRES and the Region (8228-8628) Corresponding to Part of the NS5B Gene of the Viral Genome Extracted from Positive Mosquitoes on D0 and D21

The viral RNA was extracted according to the protocol described in point 8.

Sequencing was performed from total RNA extracts from mosquitoes identified by qRT-PCR as containing the HCV genome.

The sequencing stages were in the order:
a) Amplification: The regions to be sequenced were first amplified by qRT-PCR using appropriate primers. For the region 52-271 of the IRES, the primers 1TS (SEQ ID NO: 7) and 2CH (SEQ ID NO: 6) described above were used. For the region of nucleotides 8228-8628 of the NS5b gene, the primers used were 1PR and 2PR described in the reference by Sandres-Sauné et al. in 2003.
b) Migration on gel: The amplification products were migrated in a 1% agarose gel with a molecular weight marker. At the end of migration, the amplicons were detected using EtBr. The band of interest was cut out and the sequences contained in the band extracted from the gel.
c) Cloning: Cloning was performed in the pUC18 vector. In order to be able to carry out the insertions, it was necessary to determine the restriction sites (correspondent to the restriction enzymes) present in the pUC18 vector and absent in the amplicon: the EcoRI and HindIII sites were selected. New pairs of primers were chosen from the primers described above, to which two short sequences were added on either side, one containing the EcoRI restriction site and the other the HindIII restriction site, so that the amplicon could be introduced into the vector, open and dephosphorylated.
d) Transformation: The product of cloning was introduced into TOP10 competent bacteria. The bacteria were then spread onto Petri dishes containing LB agar-Ampicillin. Finally, they were incubated overnight at 37° C.
e) Quality Control of the cloning: In order to check that the bacteria that had grown contained the insertion, a PCR was performed directly on the bacteria. The clones that had grown had been transferred using the tip of a 10 μl cone: this tip had been previously soaked in a PCR tube containing the master mix (see composition above), and then used for inoculating the Petri dish containing the LB agar-Ampicillin. At the end of the transfer stage, the PCR was started and the Petri dishes were incubated at 37° C. overnight.
f) The PCR products were migrated in a 1% agarose gel with a molecular weight marker. At the end of migration, the amplicons were detected using EtBr. The clones containing the band of the right size were amplified to produce enough plasmids for sequencing.
g) Minipreparation: The plasmids were extracted from the bacteria.
h) Sequencing: Sequencing was performed automatically by the company MWG. M13 universal primers were used. Reading was in both directions for greater accuracy and reliability.

Results

Three experiments were performed:

(i) Experiment 1: Ae. vexans females were collected in the larval stage and were tested at an age of less than 10 days. About 20 mosquitoes were fed with a suspension containing 10⁶ copies of HCV per millilitre. After raising them for 21 days, the mosquitoes were frozen and their RNA extracted. An end-point RT-PCR had positive signals corresponding to the presence of the HCV genome in 3 mosquitoes out of 5 (results not shown).

(ii)) Experiment 2: To test the genus specificity, 36 Aedes sp. and 15 Culex pipiens were fed a suspension containing 8.3×10⁵ copies of HCV per millilitre and raised for 21 days. The qRT-PCR performed on the mosquito extracts corresponding to the 21^{st} day showed the presence of the HCV genome in 33% ( 2/6) of the Aedes mosquitoes. In contrast, HCV RNA was never detected in the Culex mosquitoes (FIG. 1).

A characteristic common to RNA virus replication is the appearance of adaptive mutations. To test this, the RNA sequences of the HCV extracted from positive mosquitoes 21 days (D21) after infection were compared with day 0 samples (D0). Two regions of the HCV genome were selected: 220 nucleotides (regions 52-271) of the 5′UTR region (5′ untranslated region) and 400 nucleotides (region 8228-8628) of the part encoding the non-structural protein 5B (NSSB or RdRp [RNA dependent RNA polymerase]). No mutation was seen in the 220 nucleotides analysed from the 5′UTR region. On the other hand, 3 adaptive mutations (262Val/Ala, 265 Pro/Ser and 316 Asn/Asp) were identified among the 400 nucleotides of the RdRp corresponding to the active site (FIG. 3).

(III) Experiment 3: 124 Ae. caspius and 85 Ae. vexans were fed a suspension containing 1.2×10⁶ copies of HCV per millilitre and raised for 15 days. All the mosquitoes were dissected to separate the heads (including the salivary glands) and the bodies (including the mesentery). The presence of HCV was investigated on samples at day 0, 4, 8 and 15. To obtain greater sensitivity, the quantitative RT-PCR (qRT-PCR) was preceded by WTA (Whole Transcriptome Amplification). Under these conditions, HCV RNA was only detected in the heads of the mosquitoes on the 15^{th} day (FIG. 2). The positive signal could not be due to residual virus, since the viral genome was totally absent in the heads of the mosquitoes on the first day (D0), even when combining WTA and qPCR.

The HCV extrinsic cycle, defined as the time required by the parasite to replicate in its host, was estimated as being greater than 15 days at an ambient temperature of 25° C. At this stage, it can be hypothesised that the ingested HCV could i) pass through the mosquito's mesentery tissues, ii) replicate in other tissues, particularly in the salivary glands.

Considered together with the adaptive mutations identified, these results provide the first evidence of the competence of certain mosquitoes, in particular Ae. caspius and Ae. vexans, to replicate natural HCV.

### REFERENCES

Kuntzen, T. et al. Viral Sequence Evolution in Acute Hepatitis C Virus Infection. J. Virol. 81 (21), 11658-11668 (2007).
Lohmann, V. et al. Biochemical properties of Hepatitis C Virus NS5B RNA-dependent RNA Polymerase and identification of amino acid sequence motifs essential for enzymatic activity J. Virol. 71(11), 8416-8428 (1997).
Neumayr, G., Propst, A., Schwaighofer, H., Judmaier, G. & Vogel, W. Lack of evidence for the heterosexual transmission of hepatitis C. Q J Med. 92, 505-508 (2000).
Sandres-Sauné K. et al. Determining Hepatitis C Genotype by analyzing the sequence of the NS5B region. J. Virol. Methods 109 (2), 187-93 (2003).

1. Hepatitis C virus (HCV) NS5B protein having a point mutation in at least one of the following positions:
in the position corresponding to residue 262 of SEQ ID NO: 1; and/or
in the position corresponding to residue 265 of SEQ ID NO: 1; and/or
in the position corresponding to residue 316 of SEQ ID NO: 1, with the exception of cases where:
the residue in the position corresponding to residue 262 of SEQ ID NO: 1 is isoleucine; or
the residue in the position corresponding to residue 262 of SEQ ID NO: 1 is isoleucine and
the residue in the position corresponding to residue 316 of SEQ ID NO: 1 is cysteine.

2. HCV NS5B protein according to claim 1 wherein the protein has a point mutation in at least one of the following positions:
in the position corresponding to residue 262 of SEQ ID NO: 1, the residue in the position corresponding to residue 262 of SEQ ID NO: 1 is not isoleucine; and/or
in the position corresponding to residue 265 of SEQ ID NO: 1; and/or
in the position corresponding to residue 316 of SEQ ID NO: 1, the residue in the position corresponding to residue 316 of SEQ ID NO: 1 is not cysteine.

3. HCV NS5B protein according to claim 1 wherein:
the residue in the position corresponding to residue 262 of SEQ ID NO: 1 is not valine, leucine, isoleucine or cysteine; and/or
the residue in the position corresponding to residue 265 of SEQ ID NO: 1 is not proline; and/or
the residue in the position corresponding to residue 316 of SEQ ID NO: 1 is not asparagine, cysteine or histidine.

4. HCV NS5B protein according to claim 1 wherein:
the residue in the position corresponding to residue 262 of SEQ ID NO: 1 is alanine; and/or
the residue in the position corresponding to residue 265 of SEQ ID NO: 1 is serine; and/or
the residue in the position corresponding to residue 316 of SEQ ID NO: 1 is aspartate or aspartic acid.

5. HCV NS5B protein according to claim 4 wherein the protein has:
alanine in the position corresponding to residue 262 of SEQ ID NO: 1; and
serine in the position corresponding to residue 265 of SEQ ID NO: 1; and
aspartic acid in the position corresponding to residue 316 of SEQ ID NO: 1.

6. HCV NS5B protein according to claim 5 wherein the protein includes the sequence SEQ ID NO: 3.

7. HCV NS5B protein fragment including at least one mutation as set out in claim 1.

8. Nucleic acid sequence encoding an HCV NS5B protein according to claim 1.

9. Nucleic acid sequence according to claim 8 wherein the sequence includes the sequence SEQ ID NO: 5.

10. HCV NS5B protein according to claim 1 wherein the protein is of human origin.

11. Vector including a nucleic acid sequence according to claim 8.

12. Replicon including a nucleic acid sequence according to claim 8.

13. Host cell including a vector according to claim 11.

14. Host cell according to claim 13, wherein said cell is a cell from a mosquito.

15. Host cell according to claim 14, wherein the mosquito is the Aedes genus.

16. Nucleic acid sequence encoding the HCV NS5B protein fragment of claim 7.

17. The protein fragment of claim 7, wherein the fragment is of human origin.

18. The nucleic acid sequence of claim 8, wherein the sequence is of human origin.

19. Host cell including a replicon according to claim 12.

20. Host cell of claim 19 wherein said cell is a cell from a mosquito.
